(19) United States
(12) Patent Application Publication
(10) Pub. No.: US 2020/0107876 A1
(43) Pub. Date: Apr. 9, 2020

(54) MULTI-FUNCTION SURGICAL TRANSECTION INSTRUMENT

(71) Applicant: COVIDIEN LP, MANSFIELD, MA (US)

(72) Inventors: JAMES D. ALLEN, IV, Broomfield, CO (US); Thomas E. Drochner, Longmont, CO (US); Rachael Kabala, Fort Collins, CO (US); John A. Hammerland, III, Arvada, CO (US); Daniel A. Joseph, Golden, CO (US)

(21) Appl. No.: 16/580,695

(22) Filed: Sep. 24, 2019

Related U.S. Application Data
(60) Provisional application No. 62/740,610, filed on Oct. 3, 2018.

(51) Int. Cl. A61B 18/14

(52) U.S. Cl. CPC A61B 18/1445; A61B 2018/0063

(57) ABSTRACT

A surgical instrument includes a housing having a first handle depending therefrom and an elongated shaft. An end effector is included having first and second jaw members each having an electrically conductive plate disposed thereon. The first jaw member includes one or more electrodes disposed at a distal end thereof. A second handle operably couples to the housing and is moveable relative to actuate the jaw members to grasp tissue disposed therebetween. A first switch is activatable to supply electrical energy to the electrically conductive plates to pre-coagulate tissue. A second switch is electrically coupled to the electrode and activatable to provide electrical energy to the one or more one electrodes. A third switch is configured to supply electrical energy to tissue disposed between the electrically conductive plates. An irrigation actuator is operable to selectively supply electrically conductive fluid to the first and second jaw members proximate the electrode(s).

### CROSS REFERENCE TO RELATED APPLICATION

The present application claims the benefit of and priority to U.S. Provisional Application Ser. No. 62/740,610, filed on Oct. 3, 2018 the entire contents of which are incorporated herein by reference.

### BACKGROUND

### Technical Field

The present disclosure relates generally to the field of surgical instruments. In particular, the disclosure relates to a multi-function surgical transection device for use with hepatic-related surgical procedures.

### Background of Related Art

Surgical instruments such as electrosurgical forceps are commonly used in open and endoscopic surgical procedures to treat tissue, e.g., coagulate, cauterize, cut and/or seal tissue. The combination of mechanical clamping force and electrosurgical energy has been demonstrated to facilitate treating tissue and, specifically, sealing tissue. With respect to mechanical clamping pressure for tissue sealing, for example, it has been found that pressures within the range of about 3 kg/cm² to about 16 kg/cm² help ensure formation of effective and consistent tissue seals. Other pressures within or outside this range may be utilized for treating tissue in a different manner and/or for other purposes.

Electrosurgical forceps typically include a pair of jaw members that can be manipulated to grasp targeted tissue. More specifically, the jaw members may be approximated to apply a mechanical clamping force to the tissue, and are associated with at least one electrode to permit the delivery of electrosurgical energy to the tissue. The jaw members may be used in conjunction with a knife or an electrical cutting mechanism for cutting or transecting tissue.

Hepatic resection is a surgical procedure with many challenges due to an increased risk of bleeding and complications relating to the anatomy of the liver, i.e., complexity of the biliary and vascular anatomy of the liver. Liver transection is the most challenging part of hepatic resection and is associated with a risk of possible hemorrhage. An important factor for a better outcome is reduced blood loss due to improvements in surgical instruments and with surgical techniques. As a result, during complicated surgical procedures, e.g., hepatic transection or resection, additional surgical instruments may be used along with a surgical forceps to supplement or replace specific functions of the forceps, e.g., ultrasonic instruments, sutures, clip appliers, staplers, coagulators, etc.

Various surgical techniques have been used in the past to facilitate liver transection, so-called “clamp crushing” and the use of intraoperative ultrasound being the most prominent. In these procedures, liver parenchyma is “crushed” out of the way leaving vessels and bile ducts exposed which can then be sealed with energy, clipped, stapled, cut or treated with monopolar or bipolar energy, cold knife, etc. More recently, technological advances have led to the development of new instruments for use with liver transections, e.g., LigaSure®, TissueLink, and Aquamantys™ for example. Moreover, advances in operative techniques have also contributed to a reduction in blood loss during liver transection. These include better delineation of the transection plane with the use of intraoperative ultrasound, and better inflow and outflow control of fluids.

Typically, a combination of instruments are utilized to perform a liver transection, e.g., such as one instrument that can employ clamp crushing and another that can deliver energy, to improve the safety of liver transection. Many of these instruments utilize various types of energy modalities to coagulate tissue, seal vessels, cut and transect hepatic tissue. Other instruments use different technology to treat tissue, e.g., the liver parenchyma tissue may be fragmented with ultrasonic energy and aspirated, thus exposing vascular and ductal structures that can be ligated or clipped with titanium hemoclips.

LigaSure® (Valley Lab, Tyco Healthcare (now Medtronic, Inc.), Boulder, Colo., USA) vessel sealing instruments are another line of instrumentation designed to seal small vessels using a different principle. By a combination of compression pressure and bipolar radiofrequency (RF) energy, the various instruments apply pressure and energy to denature the proteins in the collagen and elastin and allow them to fuse together the opposing layer of denatured proteins. These instruments are effective in sealing small vessels up to 7 mm in diameter. LigaSure® in combination with a clamp crushing technique has resulted in lower blood loss and faster transection speed in minor hepatic resections compared with conventional techniques of electrical cautery or ligature for controlling vessels in the transection plane.

RF ablation (RFA) is a relatively newer technique for liver transection. A Cool-tip® RF electrode (sold by Medtronic, Inc.) is inserted along the transection plane and RF energy is applied to create overlapping cylinders of coagulated tissue, followed by transection of the coagulated liver using a simple scalpel. This device and technique has the advantage of simplicity compared with the aforementioned transection devices and techniques but tends to sacrifice too much parenchymal tissue.

### SUMMARY

As used herein, the term “distal” refers to the portion of the instrument or component thereof that is being described that is further from a user, while the term “proximal” refers to the portion of the instrument or component thereof that is being described that is closer to a user. Further, to the extent consistent, any of the aspects described herein may be used in conjunction with any of the other aspects described herein. As used herein the term “tissue” is meant to include variously-sized vessels.

Provided in accordance with aspects of the present disclosure is a surgical instrument including a housing having a first handle depending therefrom and an elongated shaft extending distally from the housing. An end effector is disposed at a distal end of the elongated shaft and includes first and second jaw members each having an electrically conductive plate disposed thereon. The respective electrically conductive plates of the first and second jaw members are disposed in vertical opposition relative to one another. The first jaw member includes one or more electrodes disposed at a distal end thereof at a position normal to the respective electrically conductive plate of the first jaw member. A second handle is operably coupled to the housing and is selectively moveable relative to the first handle to actuate the first and second jaw members between a first position wherein the first and second jaw members of the end effector assembly are disposed in a spaced apart configuration relative to one another and second position wherein the jaw members cooperate to grasp tissue disposed therebetween.

A first switch is disposed on the housing and is activatable to supply electrical energy from an electrical energy source to one or both of the electrically conductive plates of the first or second jaw members. The first switch, when activated, provides electrical energy to the respective jaw member(s) to pre-coagulate tissue. A second switch is disposed on the housing and is electrically coupled to the one or more electrodes. The second switch is activatable to provide electrical energy from the electrical energy source to the one or more electrodes for treating tissue.

A third switch is disposed on an inner facing surface of the first handle in alignment with the second movable handle such that actuation of the second movable handle activates the third switch during movement thereof. The third switch operably connects to both electrically conductive plates of the first and second jaw members and is configured to supply electrical energy from the electrical energy source to tissue disposed between the electrically conductive plates.

An irrigation actuator is operably associated with the housing and is actuatable to selectively supply electrically conductive fluid to a port defined in one or both of the first and second jaw members at a position proximate the one or more electrode(s).

In aspects according to present disclosure, the first jaw member includes first and second electrodes at the distal end thereof at a position normal to the respective electrically conductive plate of the first jaw member and the port is defined between the first and second electrodes. In other aspects, the first electrode is adapted to connect to a first polarity of the electrical energy source and the second electrode is adapted to connect to a second polarity of the electrical energy source such that, upon activation of the second switch, tissue is treated in a bipolar manner.

In yet other aspects according to the present disclosure, the irrigation actuator is movable between a first position configured to supply electrically conductive fluid to the port and a second position configured to supply suction to the port. The irrigation actuator may be configured as a toggle switch, dial, sliding tab, etc.

In yet other aspects according to the present disclosure, a trigger assembly is operably associated with the housing and includes a trigger configured to selectively advance a knife between the first and second jaw members upon actuation thereof. In still other aspects, the knife is advanceable via actuation of the trigger between a first position wherein the knife is disposed proximal the first and second electrically conductive plates of the first and second jaw members to a second position wherein the knife translates through respective channels defined within the first and second electrically conductive plates of the first and second jaw members.

In aspects according to the present disclosure, the surgical instrument further includes a rotation knob operably associated with the elongated shaft of the housing that is configured to selectively rotate relative to the housing to rotate the elongated shaft and the end effector at the distal end thereof.

In yet other aspects according to the present disclosure, the first jaw member includes a monopolar electrode at the distal end thereof at a position normal to the respective electrically conductive plate of the first jaw member and one or more ports are defined within the monopolar electrode. The monopolar electrode may be ball-shaped or any other conventional shape depending upon a particular purpose.

In still other aspects according to the present disclosure, the third switch is configured to include tactile feedback or an audible tone to advise the user prior to activation of electrical energy. The distal portions of the first and second jaw members may be curved along the same plane relative to a transverse axis defined across the end effector to facilitate visualization when plunging into parenchyma.

Provided in accordance with other aspects of the present disclosure is a surgical instrument including a housing having a first handle depending therefrom and an elongated shaft extending distally from the housing. An end effector is disposed at a distal end of the elongated shaft and includes first and second jaw members each having an electrically conductive plate disposed thereon. The respective electrically conductive plates of the first and second jaw members are disposed in vertical opposition relative to one another. The first jaw member includes first and second electrodes disposed at a distal end thereof at a position normal to the respective electrically conductive plate of the first jaw member.

A second handle is operably coupled to the housing and is selectively moveable relative to the first handle to actuate the first and second jaw members between a first position wherein the first and second jaw members of the end effector assembly are disposed in a spaced apart configuration relative to one another and second position wherein the jaw members cooperate to grasp tissue disposed therebetween.

A first switch is disposed on the housing and is activatable to supply electrical energy from an electrical energy source to one or both of the electrically conductive plates of the first or second jaw members. The first switch, when activated, provides electrical energy to the electrically conductive plate of the respective one or both jaw members to pre-coagulate tissue. A second switch is disposed on the housing and is electrically coupled to the electrode. The second switch is activatable to provide electrical energy from the electrical energy source to the first and second electrodes. The first electrode is adapted to connect to a first polarity of the electrical source energy and the second electrode is adapted to connect to a second polarity of the electrical energy source, such that upon activation thereof, tissue is treated in a bipolar manner.

A third switch is disposed on an inner facing surface of the first handle in alignment with the second movable handle such that actuation of the second movable handle activates the third switch during movement thereof. The third switch is operably connected to both electrically conductive plates of the first and second jaw members and is configured to supply electrical energy from the electrical energy source to tissue disposed between the electrically conductive plates.

### BRIEF DESCRIPTION OF THE DRAWINGS

Various aspects and features of the present disclosure are described herein with reference to the drawings, wherein like reference numerals identify similar or identical components, and wherein:

FIG. 1 is a perspective view of a surgical instrument in accordance with the present disclosure having two opposing jaw members at a distal end thereof;

FIG. 2A is an enlarged, side view of a housing of the surgical instrument showing a suction irrigation control lever, a coagulation switch and a slow close bipolar switch;

FIG. 2B is a rear perspective view of the surgical instrument of FIG. 1 showing the slow close bipolar switch and a vessel sealing, in-line activation switch;

FIG. 3A is an enlarged, end view of a distal tip of one of the jaw members having a pair of bipolar electrodes integrated therewith;

FIG. 3B is an enlarged, end view of the distal tip of one of the jaw members having a monopolar electrode integrated therewith; and

FIG. 4 is an enlarged, side view of the pair of jaw members disposed in an open position illustrating the curvature of the distal tip of the jaw members.

### DETAILED DESCRIPTION

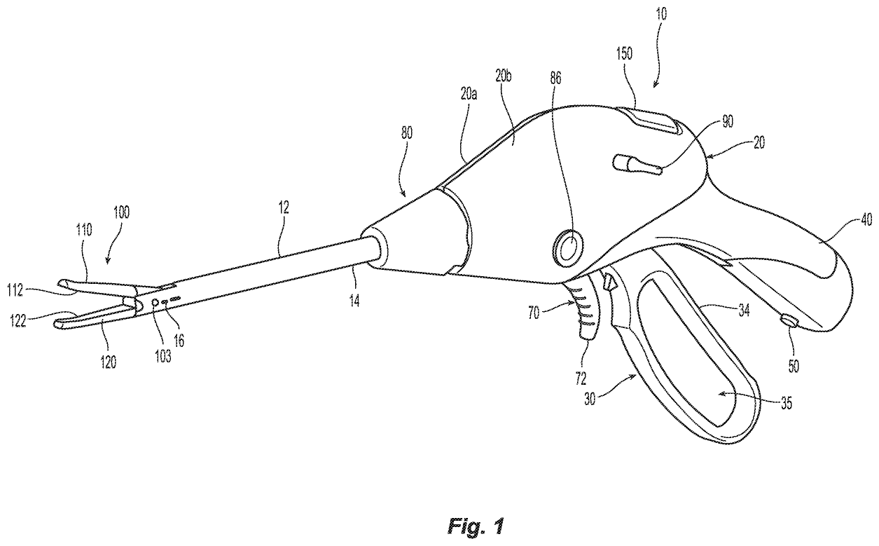

Turning to FIG. 1, a surgical instrument 10 configured for use in accordance with the present disclosure is shown generally including a housing 20 that supports various actuators and switches, e.g., a movable handle 30, a trigger assembly 70, a rotation knob 80, a bipolar pre-coagulation activation switch 150, a suction irrigation lever 90, a coagulation activation switch 86 and a vessel sealing activation switch 50. Although illustrated and described herein as an electrosurgical forceps configured for use in laparoscopic or endoscopic surgical procedures, the aspects and features of the present disclosure are equally applicable for use with other surgical instruments configured for use in traditional open surgical procedures and/or laparoscopic or endoscopic surgical procedures. For the purposes herein, instrument 10 is generally described.

Housing 20 of instrument 10 is constructed of a first housing half 20a and a second housing half 20b that are configured to support an elongated shaft 12 at a proximal end 14 thereof. Housing halves 20a, 20b may be constructed of sturdy plastic, or other suitable material, and may be joined to one another by adhesives, ultrasonic welding, or other suitable assembly process. Housing 20 supports a stationary handle 40, movable handle 30, trigger assembly 70, and rotation knob 80. Movable handle 30, as detailed below, is operable to move a pair of opposing jaw members 110 and 120 of an end effector assembly 100 disposed at a distal end 16 of elongated shaft 12. Jaw members 110 and 120 are selectively movable via handle 30 between an open configuration (FIG. 1), wherein jaw members 110, 120 are disposed in spaced relation relative to one another, and a closed configuration (not shown), wherein jaw members 110, 120 are approximated relative to one another.

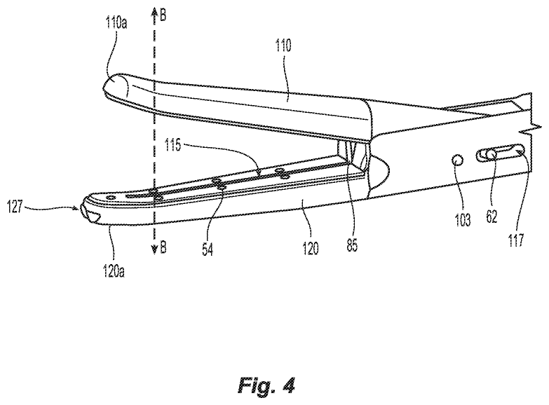

More specifically, compression of movable handle 30 towards stationary handle 40 serves to move a drive assembly (not shown) which, in turn, moves the jaw members 110, 120 of the end effector assembly 100 to the closed configuration and return of movable handle 30 away from stationary handle 40 serves to move the jaw members 110, 120 of the end effector assembly 100 back to the open configuration. Trigger assembly 70 is operable to extend and retract a knife blade 85 (see FIG. 4) between jaw members 110, 120 when the end effector assembly 100 is in the closed configuration. Rotation knob 80 serves to rotate elongated shaft 12 and end effector assembly 100 relative to housing 20.

Each jaw member 110, 120 includes an electrically conductive plate 112, 122, respectively, disposed thereon that is configured to conduct electrical energy to tissue when held therebetween. One or both electrically conductive plates, e.g., electrically conductive plate 122, includes a knife channel, e.g., knife channel 115, defined therein that is configured to allow selective reciprocation of the knife blade 85 therein upon actuation (e.g., squeezing) of a trigger 72 of trigger assembly 70.

To electrically control the jaw members 110, 120 and the various energy modalities associated therewith, housing 20 supports a variety of switches that provide different energy modalities to different electrodes disposed on the jaw members 110, 120. More particularly, switch 150 is disposed towards the proximal portion of housing 20 and is configured to provide bipolar energy to electrically conductive plates 112, 122 to pre-coagulate tissue prior to further tissue treatment by one of the other modalities as explained below. Activation of switch 150 provides a first energy polarity from a generator (not shown) to electrically conductive plate 112 and a second energy polarity to electrically conductive plate 122 such that electrical current passes through tissue when disposed between jaw members 110, 120. Pre-coagulating tissue and slowly closing (e.g., “slow close”) the jaw members 110, 120 effectively pre-heats the tissue to facilitate further treatment. More particularly, the jaw members 110, 120 are slowly closed while activating the forceps 10. The speed of the jaw closure is closely regulated by the surgeon to maintain a blanched are of parenchyma around the jaw members 110, 120. Once completely closed the forceps is again activated to complete a seal. As a result of this technique liver parenchyma is crushed between the jaw members 110, 120 with sufficient coagulation and the liver can be divided with minimal bleeding from the liver parenchyma.

Slow close pre-coagulation allows the user to apply energy to tissue before the jaw members 110, 120 are completely closed. This is helpful in solid organ surgery, and potentially large tissue bundles, as the tissue between the jaw members 110, 120 may heat up enough to coagulate small vessels and parenchyma. Therefore, as the surgeon continues to move the jaw members 110, 120 into the closed position, there will be reduced bleeding. This may eliminate the need to address any potential “oozing” that may occur as well as create a cleaner operating field for better visualization.

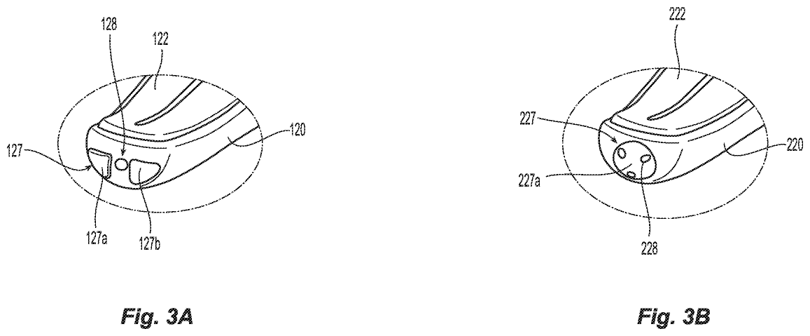

Switch 86 is disposed on one or both sides 20a, 20b of housing 20 and is configured to supply energy to the distal tip 127, 227 of jaw member 120 depending on the configuration of the instrument 10, e.g., jaw member 120 may include a bipolar tip option (FIG. 3A) or a monopolar tip option (FIG. 3B) which is each activated via switch 86. More particularly and as best illustrated by FIG. 3A, the distal tip 127 of jaw member 120 may include a pair of electrodes 127a and 127b that cooperate to supply bipolar energy to tissue disposed proximate tip 127. Electrode 127a may be connected to an energy source (not shown) that supplies a first energy polarity to electrode 127a and electrode 127b may be connected to the energy source to supply a second energy polarity to electrode 127b such that an electrical current flows through tissue disposed therebetween. The energy source may be the same or a different energy source that energizes electrically conductive plates 112, 122.

An irrigation/suction port 128 is defined between the electrodes 127a, 127b (or proximate the electrodes 127a, 127b) to supply saline (or any other conductive medium) to the area proximate tip 127 to aid coagulation or enhance the coagulation effect. When used as an irrigation/suction port, fluids may be safely evacuated to improve visualization and assist in controlling the buildup of heat. As explained in more detail below, lever 90 on housing 20 is configured to control the delivery of fluid, e.g., saline, or the release of suction to port 128.

As best illustrated by FIG. 3B, jaw member 120 may include a distal tip 227 that includes a monopolar electrode 227a that is energizable via switch 86 to provide monopolar energy to tissue proximate the distal tip 227. Electrode 227a may be ball-shaped and may be connected to an energy source (not shown) that supplies a first energy polarity to electrode 227a and a return electrode, e.g., a return pad (not shown), may be connected to the energy source to supply a second energy polarity to the return electrode such that an electrical current flows through tissue proximate the distal tip 227. Energy is concentrated at the tip electrode 227a and is generally dispersed at the return pad electrode due to the size differential between the two electrodes. An irrigation port or ports 228 may be disposed proximate electrode 227a to supply saline (or any other conductive medium) to the area proximate tip 227 to aid coagulation. Again, lever 90 on housing 20 is configured to control the delivery of fluid, e.g., saline, or the release of suction to port(s) 228.

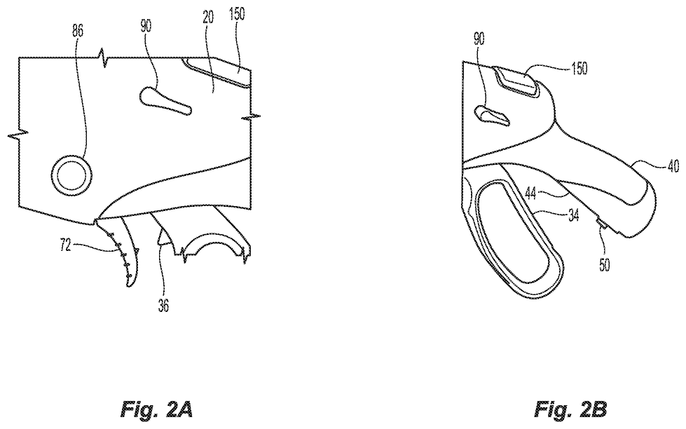

Referring to FIGS. 2A and 2B and as mentioned above, lever 90 is disposed on one or both sides of housing 20 and is operable to supply fluid or suction to the operative site. Although the irrigation/suction ports 128, 228 are disposed on the distal tips 127, 227 of instrument 10, one or more ports may be disposed at different locations on the end effector assembly 100. Lever 90 is selectively movable from a neutral position (i.e., a position where neither irrigation or suction is supplied) to a first position where irrigation is supplied to one or more ports, e.g., port 128, or a second position where suction is supplied to one or more ports, e.g., port 128. For example and as shown in FIG. 2A, a user can actuate lever 90 in a first direction (upwardly) to initiate the flow of fluid to one or more ports, e.g., port 128, and a user can actuate lever 90 in a second direction (downwardly) to initiate the flow of suction to one or more ports, e.g., port 128. The user can toggle between positions as needed during a surgical procedure.

FIG. 2B shows activation switch 50 that is configured to provide electrosurgical energy to the end effector assembly 100 for sealing tissue when activated. Switch 50 is positioned as an in-line activation switch that is only activatable when the jaw members 110, 120 are closed about tissue. More particularly, switch 50 is disposed on a distal portion 44 of handle 40 angular registration with a proximal portion 34 of movable handle 30 such that during the range of motion from an open position (corresponding to the jaw members 110, 120 being disposed in a spaced apart position) to a closed position (corresponding to the jaw members 110, 120 being disposed in an approximated position) the proximal portion 34 of movable handle 30 activates the switch 50.

A tactile sensation or audible tone (or both) may be operably associated with movable handle 30 or the switch 50 to advise the user prior to activation of energy. As mentioned above, upon activation, switch 50 is configured to supply electrosurgical energy to tissue disposed between electrically conductive plates 112, 122 of jaw members 110, 120, respectively, to effectively seal tissue. One or more algorithms associated with sealing technology may be employed to accomplish this purpose, e.g., Medtronic's LigaSure® algorithm, used with its proprietary vessel sealing generators, e.g., Force Triad™, Force FX™, Force EZ™, etc. and line of vessel sealing instruments, e.g., LigaSure Atlas™, LigaSure Precise™, LigaSure Impact™ LigaSure Advance™, LigaSure Maryland™, LigaSure Dolphin Tip, LigaSure Exact, etc.

Referring to FIG. 4, as mentioned above, end effector assembly 100 includes jaw members 110, 120 each having an electrically conductive plate 112, 122 disposed thereon which cooperates to engage and treat tissue when one or more switches are activated, e.g., switch 150 and/or switch 50. The distal-most end of each jaw member 110, 120 includes a tip portion 110a and 120a, respectively, that are each configured to curve in the same direction (coplanar) along a transverse axis “B-B” defined through the jaw members 110, 120. The curved tip portions 110a, 120a are designed to aid visualization of tissue during use, e.g., aid visualizing tissue when plunging the tip portions 110a, 120a into parenchyma tissue. The tip portions 110a, 120a are also designed to help the surgeon visualize the location of the tips 110a, 120a when plunging the tips 110a, 120a into parenchyma. The jaw members 110, 120 may also be configured to close in a generally parallel fashion which optimizes the jaw members 110, 120 for transection parenchyma while leaving larger vessels and structures undamaged.

Jaw members 110, 120 are pivoted about a pivot pin 103 and relative to the distal end 16 of elongated shaft 12 between the open configuration (FIG. 4) and the closed configuration (not shown). In the closed configuration of end effector assembly 100, electrically-conductive plates 112, 122 of respective jaw members 110, 120 provide a clamping pressure to the tissue grasped therebetween. Also, in the closed configuration, a gap distance may be maintained between electrically-conductive plates 112, 122 by one or more stop members 54 disposed on either or both electrically-conductive plates 112, 122. The gap distance may be in the range of about 0.001 inches to about 0.010 inches or higher.

A drive assembly (not shown) operably couples movable handle 30 with end effector assembly 100 such that, as noted above, movable handle 30 is operable to move jaw members 110, 120 of end effector assembly 100 between the open configuration and the closed configuration. The drive assembly may include a drive rod slidably disposed within elongated shaft 12 and operably coupled to jaw members 110, 120, e.g., via a pin 62 associated therewith and extending through oppositely-angled slots, e.g., slot 117, defined within the proximal flanges of the jaw members, e.g., jaw member 110, such that proximal sliding of drive rod and pin 62 through elongated shaft 12 moves end effector assembly 100 from the open configuration to the closed configuration. However, the opposite configuration is also contemplated, as are other mechanisms for operably coupling the drive rod with jaw members 110, 120. The drive rod and pin 62 arrangement along with the drive assembly may be optimized to allow precise surgical feel and control of the movement of the jaw members 110, 120 during specific surgical procedures, e.g., clamp-crushing, to assist in identifying internal hepatic structures.

Movable handle 30 is pivotably coupled within housing 20 via a pivot pin (not shown) and is operably coupled to the drive rod such that movable handle 30 may be manipulated to impart longitudinal motion to drive rod and pin 62. As noted above, longitudinal movement of drive rod, in turn, moves end effector assembly 100 between the open and closed configurations. During initial movement of the movable handle 30, jaw members 110, 120 meet minimal resistance as they move towards the closed condition due to an internal spring maintaining a pre-compressed condition.

Once jaw members 110, 120 are closed about tissue and/or when jaw members 110, 120 otherwise meet sufficient resistance, further pivoting of movable handle 30 towards stationary handle 40 compresses the spring which essentially acts as a force-regulator to ensure that an appropriate clamping pressure is applied to tissue grasped between jaw members 110, 120. For tissue sealing, for example, this pressure may be within the range of about 3 kg/cm² to about 16 kg/cm²; however, other suitable pressures may also be provided.

As noted above, the compression of spring enables the regulation of the clamping pressure applied to tissue grasped between jaw members 110, 120, allows the surgeon to regulate the jaw members 110, 120 during specific surgical procedures such as clamp crushing parenchyma, and enhances a surgeons “feel” when interacting with internal hepatic structures.

Referring to FIGS. 1 and 4, trigger assembly 70 may be manipulated to impart longitudinal motion to knife blade 85 (FIG. 4) to advance the knife blade 85 through knife channel(s) 115 defined within one or both of the jaw members 110, 120 (see FIG. 4). Trigger assembly 70 includes trigger 72 that is pivotally supported in housing 20 via a pivot pin (not shown) and is operably coupled to the knife blade 85. A spring (not shown) may be included that is configured to bias the knife blade 85 towards a retracted or proximal-most position, wherein knife blade 85 is positioned proximally of jaw members 110, 120, and trigger 72 is disposed in an un-actuated position. Upon actuation of trigger 72, e.g., upon pivoting of trigger 72 towards movable handle 30, knife blade 85 is advanced distally between jaw members 110, 120 to cut tissue grasped therebetween.

By combining the various electrical modalities and algorithms associated with the above identified switches 150, 86 and 50 along with the placement of various electrodes, e.g., 127, 227, or electrically conductive surfaces, e.g., 112, 122, on the end effector assembly 100, along with the unique shape of the distal ends 110a, 120a of the jaw members 110, 120, and the precise control of the movement of the jaw members 110, 120, a surgeon can utilize one instrument 10 for various types of hepatic surgeries, e.g., transection of liver parenchyma.

The various embodiments disclosed herein may also be configured to work with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients.

For a detailed description of exemplary medical work stations and/or components thereof, reference may be made to U.S. Patent Application Publication No. 2012/0116416, and PCT Application Publication No. WO2016/025132, the entire contents of each of which are incorporated by reference herein.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

What is claimed is:

1. A surgical instrument, comprising:
a housing having a first handle depending therefrom;
an elongated shaft extending distally from the housing;
an end effector disposed at a distal end of the elongated shaft, the end effector including first and second jaw members each having an electrically conductive plate disposed thereon, the respective electrically conductive plates of the first and second jaw members disposed in vertical opposition relative to one another, the first jaw member including at least one electrode disposed at a distal end thereof at a position normal to the respective electrically conductive plate of the first jaw member;
a second handle operably coupled to the housing and selectively moveable relative to the first handle to actuate the first and second jaw members between a first position wherein the first and second jaw members of the end effector assembly are disposed in a spaced apart configuration relative to one another and second position wherein the jaw members cooperate to grasp tissue disposed therebetween;
a first switch disposed on the housing and activatable to supply electrical energy from an electrical energy source to at least one of the electrically conductive plates of the first or second jaw members, the first switch when activated providing electrical energy to the respective at least one jaw member to pre-coagulate tissue;
a second switch disposed on the housing and electrically coupled to the electrode, the second switch activatable to provide electrical energy from the electrical energy source to the at least one electrode for treating tissue;
a third switch disposed on an inner facing surface of the first handle in alignment with the second handle such that actuation of the second handle activates the third switch during movement thereof, the third switch operably connects to both electrically conductive plates of the first and second jaw members and is configured to supply electrical energy from the electrical energy source to tissue disposed between the electrically conductive plates; and
an irrigation actuator operably associated with the housing and actuatable to selectively supply electrically conductive fluid to a port defined in at least one of the first and second jaw members at a position proximate the at least one electrode.

2. The surgical instrument according to claim 1 wherein the first jaw member includes first and second electrodes at the distal end thereof at a position normal to the respective electrically conductive plate of the first jaw member and the port is defined between the first and second electrodes.

3. The surgical instrument according to claim 2 wherein the first electrode is adapted to connected to a first polarity of the electrical energy source and the second electrode is adapted to connect to a second polarity of the electrical energy source such that, upon activation of the second switch, tissue is treated in a bipolar manner.

4. The surgical instrument according to claim 1 wherein the irrigation actuator is movable between a first position configured to supply electrically conductive fluid to the port and a second position to configured to supply suction to the port.

5. The surgical instrument according to claim 4 wherein the irrigation actuator is a toggle.

6. The surgical instrument according to claim 1 further comprising:
a trigger assembly operably associated with the housing, the trigger assembly including a trigger configured to selectively advance a knife between the first and second jaw members upon actuation thereof.

7. The surgical instrument according to claim 6 wherein the knife is advanceable via actuation of the trigger between a first position wherein the knife is disposed proximal the first and second electrically conductive plates of the first and second jaw members to a second position wherein the knife translates through respective channels defined within the first and second electrically conductive plates of the first and second jaw members.

8. The surgical instrument according to claim 1 further comprising a rotation knob operably associated with the elongated shaft of the housing, the rotation knob selectively rotatable relative to the housing to rotate the elongated shaft and the end effector at the distal end thereof.

9. The surgical instrument according to claim 1 wherein the first jaw member includes a monopolar electrode at the distal end thereof at a position normal to the respective electrically conductive plate of the first jaw member and at least one port is defined within the monopolar electrode.

10. The surgical instrument according to claim 1 wherein the monopolar electrode is ball-shaped.

11. The surgical instrument according to claim 1 wherein the third switch is configured to include tactile feedback or an audible tone to advise the user prior to activation of electrical energy.

12. The surgical instrument according to claim 1 wherein distal portions of the first and second jaw members are curved along the same plane relative to a transverse axis defined across the end effector.

13. A surgical instrument, comprising:
a housing having a first handle depending therefrom;
an elongated shaft extending distally from the housing;
an end effector disposed at a distal end of the elongated shaft, the end effector including first and second jaw members each having an electrically conductive plate disposed thereon, the respective electrically conductive plates of the first and second jaw members disposed in vertical opposition relative to one another, the first jaw member including first and second electrodes disposed at a distal end thereof at a position normal to the respective electrically conductive plate of the first jaw member;
a second handle operably coupled to the housing and selectively moveable relative to the first handle to actuate the first and second jaw members between a first position wherein the first and second jaw members of the end effector assembly are disposed in a spaced apart configuration relative to one another and second position wherein the jaw members cooperate to grasp tissue disposed therebetween;
a first switch disposed on the housing and activatable to supply electrical energy from an electrical energy source to at least one of the electrically conductive plates of the first or second jaw members, the first switch, when activated, providing electrical energy to the electrically conductive plate of the respective at least one jaw member to pre-coagulate tissue;
a second switch disposed on the housing and electrically coupled to the electrode, the second switch activatable to provide electrical energy from the electrical energy source to the first and second electrodes, wherein the first electrode is adapted to connect to a first polarity of the electrical source energy and the second electrode is adapted to connect to a second polarity of the electrical energy source, such that upon activation thereof, tissue is treated in a bipolar manner; and
a third switch disposed on an inner facing surface of the first handle in alignment with the second handle such that actuation of the second handle activates the third switch during movement thereof, the third switch operably connected to both electrically conductive plates of the first and second jaw members and configured to supply electrical energy from the electrical energy source to tissue disposed between the electrically conductive plates.

14. The surgical instrument according to claim 13 further comprising:
a trigger assembly operably associated with the housing, the trigger assembly including a trigger configured to selectively advance a knife between the first and second jaw members upon actuation thereof.

15. The surgical instrument according to claim 14 wherein the knife is advanceable via actuation of the trigger between a first position wherein the knife is disposed proximal the first and second electrically conductive plates of the first and second jaw members to a second position wherein the knife translates through respective channels defined within the first and second electrically conductive plates of the first and second jaw members.

16. The surgical instrument according to claim 13 further comprising a rotation knob operably associated with the elongated shaft of the housing, the rotation knob selectively rotatable relative to the housing to rotate the elongated shaft and the end effector at the distal end thereof.

17. The surgical instrument according to claim 13 wherein the third switch is configured to include tactile feedback or an audible tone to advise the user prior to activation of electrical energy.
